(19) United States
(12) Patent Application Publication
(10) Pub. No.: US 2023/0166097 A1
(43) Pub. Date: Jun. 1, 2023

(54) STRETCHABLE TUBULAR DEVICE AND USE THEREOF AS A COUNTERPULSATION DEVICE

(71) Applicant: ECOLE POLYTECHNIQUE FEDERAKE DE LAUSANNE (EPFL), Lausanne (CH)

(72) Inventors: Yoan CIVET, Publier (FR); Yves PERRIARD, Neuchâtel (CH); Jonathan CHAVANNE, Châtel-St-Denis (CH); Morgan ALMANZA, Massy (FR); Thomas MARTINEZ, Neuchâtel (CH)

(21) Appl. No.: 17/922,526

(22) PCT Filed: Apr. 7, 2021
(86) PCT No.: PCT/IB2021/052895

§ 371 Date: Oct. 31, 2022

(30) Foreign Application Priority Data
Apr. 29, 2020 (EP) 20171968.9

(51) Int. Cl. A61M 60/148; A61M 60/486; A61M 60/531

(52) U.S. Cl. CPC A61M 60/148; A61M 60/486; A61M 60/531

(57) ABSTRACT

The present invention is related to a stretchable tubular device (1) comprising at least one layer (Lx) of a stretchable polymer, a power supply (2) and a set of electrodes (3a, 3b) connected to said power supply (2). The power supply can supply at least a first level of voltage (V1) to the electrodes so as to modify the natural force (F0) of the stretchable layers to a modified force (F1). The present invention also covers a process for manufacturing such a tubular device and its use as a medical implant.

### TECHNICAL DOMAIN

The present invention concerns a device adapted for cardiac assistance. In particular, the present device can be used as a implant, on an artery, and help the cardiac efforts.

### RELATED ART

Heart failure (HF) is defined by the American Heart Association and American College of Cardiology as “a complex clinical syndrome that can result from any structural or functional cardiac disorder that impairs the ability of the ventricle to fill with or eject blood’ (Hunt et al. 2009). HF is a major health problem affecting more 5.1 million patients in USA and more than 23 million worldwide (Roger 2013). HF is strongly related to age and, due to the ageing population, the prevalence is expected to rise in the future (Park and Choi 2016). Although heart transplant is a gold standard for severe heart failure, there is a need of alternative effective therapies due to the shortage of heart donors.
The first successful attempt to treat HF with an artificial assist devices in humans dates back to 1966 (DeBakey 1971). Despite the efforts of cardiac surgeons and biomedical engineers to develop and improve cardiac assist devices, the performances of these devices remain still limited. Notably, none of the devices which are commercially available are fully implantable (Sen et al. 2016, Eisen 2019)
Technological solutions to assist the heart can be divided into two big families: ventricular assist devices (VADs) (including total artificial heart, TAH) and aortic counterpulsation devices.

VADs are widely used in clinical setting as destination therapy, bridge to decision, bridge to recovery, bridge to transplantation (Loor and Gonzalez-Stawinski 2012). Current VADs are based on rotary pumps (axial and centrifugal pumps) characterized by a single rotating element which ensures a constant flow (Sen et al. 2016). The rotary pumps have replaced the pulsatile pumps of the 1990s because, compared to the latter, they are significantly simpler (lower number of parts which could fail), smaller in size (less invasive surgery required) and they are characterized by smaller/faster transcutaneous drivelines (electric rather than pneumatic) (Agarwal and High 2012). The main advantages of current VADs are associate to: i) mechanical reliability/possibility of long-term application, ii) fast response (due to electric actuation) and iii) small size (which can be advantageous for the less invasiveness of the surgery and for their potential use in fully implanted pumps). The main limitations of VADS are related to the high risk of haemolysis and thrombosis, due to the high shear generated by the rotating parts (patients must use anti-coagulant during their lifetime (Heilmann et al. 2009) and the lack of pulsatile flow. Despite the existence of conflicting findings, pulsatile flow is usually preferred to continuous flow as it favours optimal capillary perfusion (Barić 2014)

Aortic-counterpulsation devices can be subdivided in intra-aortic (de Waha et al. 2014, Santa-Cruz, Cohen and Ohman 2006), extra-aortic (Schulz et al. 2016, Davies et al. 2005, Sherwood et al. 2003) and para-aortic (Lu et al. 2011) counterpulsators depending on their location within the aorta. While intra-aortic counterpulsation (i.e. intra aortic balloon pump, IABP) has been widely used in clinical settings for the last 40 years, the applications of extra and para/aortic have been very limited. The basic principle of counterpulsation is similar for all devices: it allows an increase of the coronary blood flow (during diastole) and unloading of the left ventricle (during systole) (de Waha et al. 2014). To this end, a balloon is inflated during the diastolic phase of the heart (which translates into augmentation of the peak diastolic pressure) while presystolic deflation of the balloon decreases the afterload (the pressure against which the heart must pump during systole) (de Waha et al. 2014, Santa-Cruz et al. 2006). IABP is mainly used in high risk patients with severe left ventricular dysfunction as bridge to other solutions (e.g. VADs). The main advantages of IABPs are: i) simplicity to implant (access from femoral artery) ii) pulsatile flow is preserved and ii) anti-coagulation therapy is not required (Pucher et al. 2012). The main complications related to IABP are limb and mesenteric ischemia, bleeding and haemorrhage and infections (Pucher et al. 2012). These complications are associated to the direct arterial access (which also forces the patient to lie in bed) and the size of the pneumatic drivelines (comparing to electric lines). Moreover pneumatic actuation, mandatory for both extra and intra aortic counterpulsation devices can introduce uncontrollable delays (time for balloon inflation/deflation) and requires a huge external pneumatic systems and connection through the skin. This is incompatible with a permanent lightweight solution.

Examples of inflation balloons mentioned above and their use as counterpulsation device are given in the following documents “Ambulatory extra-aortic counterpulsation in patients with moderate to severe chronic heart failure”, Abraham et al. JAAC, 2014, Vol 2, N°5.

“Chronic extra-aortic balloon counterpulsation: first-in-human pilot study in end-stage heart failure”, Hayward et al. J. Heart Lung Transplant. 2010, 29, 1427-32. Solutions based on electroactive polymers have been investigated such as those of U520160144091. Such devices are however limited to active contractions and may not provide an optimal solution. There is thus a room to develop alternative solutions, which limits or avoids the drawbacks above-mentioned. Short disclosure of the invention

An aim of the present invention is the provision of an alternative device usable as a counterpulsation device and compatible with a permanent lightweight solution.

A further aim of the present invention is the provision of a counterpulsation devices which allows to better control or eliminate potential delays in the activation of the device.

Another object of the present invention is to provide a counterpulsation device which is exempt from mechanical mobile pieces. It is in particular an object of the invention to store energy resulting from the natural artery pressure and to release energy without mobile and mechanical pieces.

It is a further object to provide a counterpulsation device which mimics the natural properties of an artery, in particular the aorta. It is more particularly an aim to provide a counterpulsation device which passively mimics the natural properties of an artery such as the aorta.

It is also an aim of the present invention to provide a method of controlling the pressure of flowing liquid using the disclosed device. In particular, the liquid is blood and the device is used to regulate the blood pressure.

The present invention further provides a process for manufacturing the disclosed device.

According to the invention, these aims are attained by the device, the method and the kit described in the independent claims, and further detailed through the corresponding dependant claims.

### SHORT DESCRIPTION OF THE DRAWINGS

Exemplar embodiments of the invention are disclosed in the description and illustrated by the following drawings:

FIG. 1a: Schematic representation of the stretchable tubular device 1 in absence of external forces. (The two layers L1, L2 are arbitrarily represented).

FIG. 1b: Schematic representation of the stretchable tubular device 1 under the application of an expending force. (only 1 layer L1 is represented for simplicity)

FIG. 1c: Schematic representation of the stretchable tubular device 1 retrieving its native diameter D0 after the expending force has decreased. (only 1 layer L1 is represented for simplicity)

FIG. 2: Schematic view of the tubular device of the present invention used as a counterpulsation device placed on the aorta, during the systole period and during the diastole period

FIGS. 3a, 3b, 3c: Schematic view of the process steps for the manufacturing of the stretchable device 1.

### DETAILED DESCRIPTION

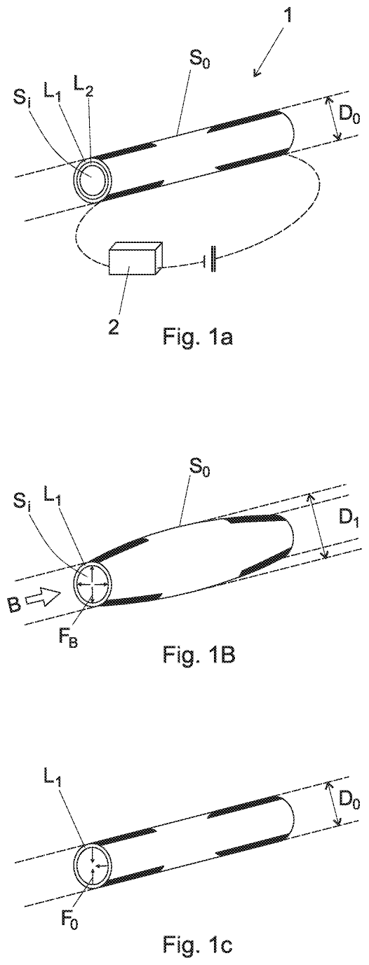

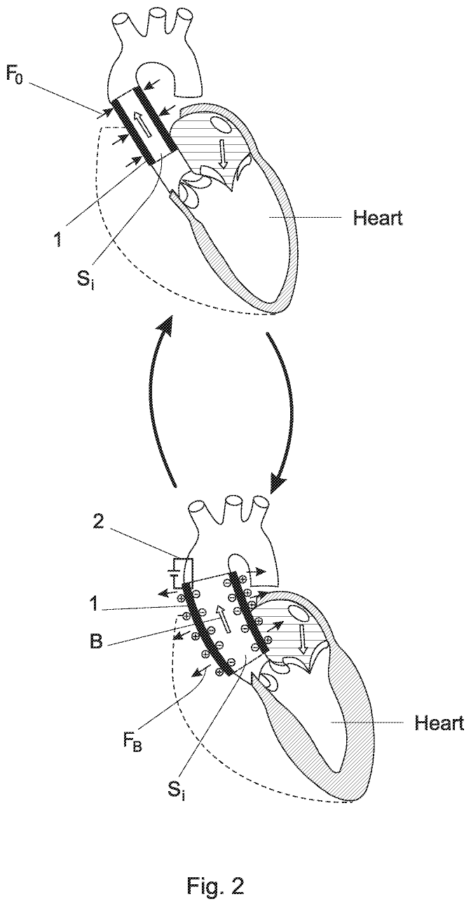

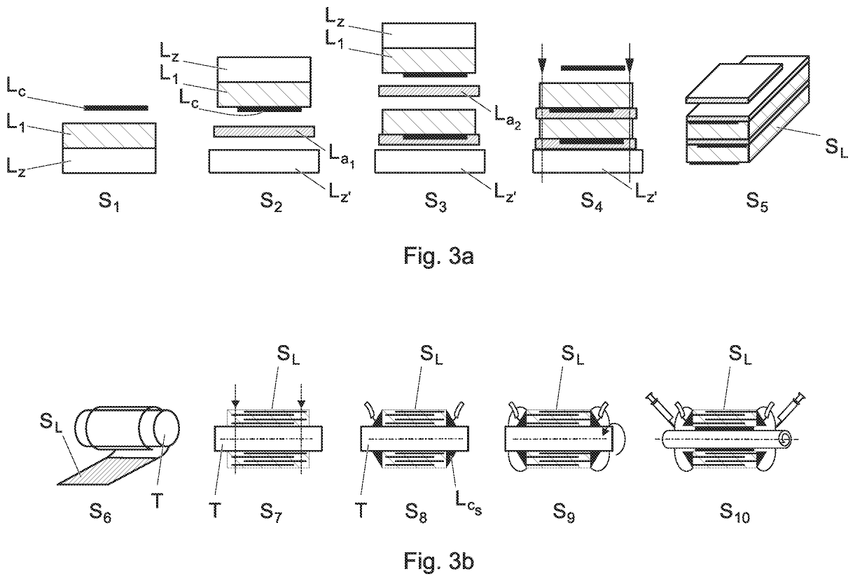

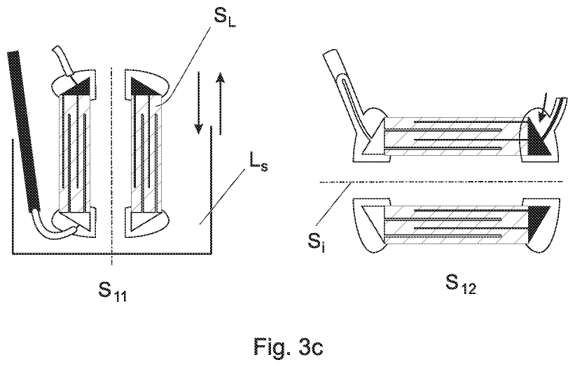

FIGS. 1a, 1b, 1c show a schematic view of the stretchable tubular device 1 according to the present invention. The stretchable tubular device 1 comprises one or several layers L1, L2 . . . Lx, stacked in a tubular arrangement having an inner space Si and an outer space So. The inner space Si of the tubular device 1 may serve as a duct through which a liquid B can flow. The nature of the flowing liquid B has no importance. It can be for example pure water, an aqueous solution such as a physiological solution, an organic medium such as blood or any other liquid. The flowing liquid B exerts an expending force FB against the internal wall of the tubular device 1. This expending force FB, which is schematically directed from the inner space Si toward the outer space So of the tubular device 1, depends for example on the inner pressure of the flowing liquid B. The higher the inner pressure is, the stronger is the expanding force FB applied to the tubular device 1. The expending force FB thus corresponds to the internal pressure of the tubular device 1 when a liquid B is flowing through. Since the tubular device 1 is stretchable, the expending force FB of the flowing liquid B tends to increase its diameter D above its native diameter D0. The native diameter D0 should be understood here as the diameter of the tubular device 1 in absence of any external forces applied to the stretchable tubular device 1, and in particular in absence of the expending force FB of the flowing liquid B.

The tubular device 1 has an inherent visco-elasticity E0. The inherent visco-elasticity E0 should be understood as intrinsically resulting from the physical chemical properties of the layers Lx of the tubular device 1 in absence of any external perturbation, and in particular in absence of electrical perturbations. When the diameter D of the tubular device 1 is extended larger than its native diameter D0, then its inherent visco-elasticity exerts a natural force F0 tending to recover its native diameter D0. In other words, the stretchable tubular device 1, when undergoing the expending force FB of the flowing liquid B, opposes its natural force F0 which reduces the expansion of its diameter D. The inherent visco-elasticity E0 thus exerts a natural force F0 directed toward the inner space Si of the tubular device 1 when its diameter is extended to a diameter D1 larger than its native diameter D0. The natural force F0 is thus contrary to the expending force FB of the flowing liquid B. The natural force F0 of the tubular device 1 depends from parameters such as the length of the stretchable tubular device 1, the nature of the stretchable layers Lx of polymers, their number x, their corresponding thickness Tx. The inherent visco-elasticity E0 may be predetermined according to the application of the stretchable tubular device 1. It may range for example between around 50 mmHg and around 200 mmH.g. It is noted here that the natural force is equivalent to a pressure since it is opposed to the internal pressure of the flowing liquid B, and that it can be determined with mmHg units.

Each one of the opposite faces of a given layer Lx is provided with an electrode or a set of electrodes 3a, 3b. Such electrodes may for example take the form of conductive layer Lc, such as a carbon layer or any other known electrically conductive material. The thickness of the electrodes Tx may be comprised between around 10 nm and around 100 μm. It is typically in the order of around 5 to 10 μm. The electrodes may advantageously be stretchable, at least in some instance, so as to resist to the stretching of the polymer layers Lx to which they are connected with. The electrodes 3a, 3b can thus induce a voltage difference between the two opposite faces of a layer Lx. One or several voltage level Vn, n being the number of voltage levels, can be applied to the stretchable tubular device 1. At least a first level of voltage V1 may be applied to the stretchable tubular device 1. The application of a first voltage level V1 modifies the visco-elasticity of the stretchable layers Lx of polymers, which then takes a first modified visco-elasticity E1 value. It results that the natural force F0 is also modified to a first modified force F1. When the diameter D of the stretchable tubular device 1 is extended above its native diameter D0, then the first modified force F1, different to the natural force F0, tends to recover the native diameter D0. As the natural force F0, the modified forces of the tubular device 1 are directed toward the inner space Si of the stretchable tubular device 1. The first modified force F1 is preferably lower than the natural force F0 of the stretchable tubular device 1. It is however not excluded that a higher force arises under the voltage application, even though this alternative is not preferred.

Under the application of a first voltage level V1, the first modified force F1, different from the natural force F0 applies. In case the first modified force F1 is lower than the natural force F0, the resistance to an expending force FB of a flowing liquid B decreases. Under a given pressure of the flowing liquid B, the diameter of the tubular device 1 tends to increase to a first modified diameter D1 under the application of the first level of voltage V1 which is higher than the diameter obtained without any voltage application. In case a second voltage V2 is applied to the stretchable tubular device 1, higher than the first level of voltage V1, then the diameter further increases to a second modified diameter D2, larger than the first modified diameter D1 for the same given expending force F0. In other words, the stretchable tubular device 1 opposes less force to the expending force FB of the flowing liquid B. This limits the pressure increase inside the stretchable tubular device 1, when the inner pressure of the flowing liquid B increases. It is here noted that the application of a voltage does not actively expends the stretchable tubular device 1, but merely modifies its stretching properties.

The difference between the natural force F0 and the first modified force F1 may be determined according to the needs. For example, a difference of 3 to 20 mmHg, or 5 to 10 mmHg may be obtained when applying a first level of voltage V1. Such a first level of voltage V1 may be comprised between 1000 V and 20 kV, typically between around 6 kV to 12 kV.

The force of the stretchable tubular device 1, either its natural force F0 or one of its forces F1, F2, Fn modified under the application of a voltage, depends on the number x of layers Lx and their thickness Tx. The number of layers Lx may be comprised between 2 and 100. It may be for example around 10 to 50.

A given layer Lx may have a thickness Tx of between around 100 μm or 200 μm. A given layer Lx may have a capacitance of between around 0.8 nF and 1 nF. A given layer Lx may have a sheet resistance of between around 1 KΩ and 10 kΩ. The applicable voltage may vary according to the physical and chemical properties of the layers Lx.

According to an embodiment, a given layer Lx may have a thickness Tx of around 100 μm a capacitance of around 1.6 nF and a sheet resistance of around 1 kΩ. The first voltage level V1 may be around 6 kV. According to another embodiment, a given layer Lx may have a thickness Tx of around 200 μm, a capacitance of around 0.8 nF and a sheet resistance of around 10 kΩ. The first voltage V1 may be around 12 kV.

The electrodes 3a, 3b are connected to a power supply 2, which can take any usable form. In particular, the power supply may be a battery.

The change of the visco-elastic properties is immediate, meaning it takes less than 20 ms, preferably less than 1 ms under the application of a voltage.

The power supply 2 may be adapted to alternatively apply a level of voltage V and suppress the applied voltage V at predetermined frequencies. A first level of voltage V1 may be applied for example for a duration of few milliseconds, or one second or several seconds. It may then be suppressed for a dead time of few milliseconds or one second or several seconds. When the stretchable tubular device 1 surrounds a flexible duct wherein circulates a constant flow of liquid B, the ON/OFF alternance may induce waves of pressures. In particular, when a voltage is applied, the flowing liquid increases the diameter of the stretchable tubular device 1. Once it is stopped, the natural force F0 of the stretchable device 1 is recovered and the tubular device naturally retracts, generating a wave of pressure in the flowing liquid B.

The stretchable tubular device 1 may also be used on a none-constant flow of liquid B, such as the blood flow within the body. In particular, it can surround an artery or replace a portion of an artery. The present stretchable tubular device 1 may be used as an aortic counterpulsation device for example. It can store elastic energy during the systole and release it during the diastole. A first level of voltage V1 may be applied to the stretchable tubular device 1 at the moment of the systole or shortly before the systole. Shortly before means few milliseconds to few tenth of milliseconds. Typically, a first level of voltage V1 may be applied around 50 ms before the systole. The voltage may then be stopped at the moment of the diastole or shortly after. The recovered natural force F0 of the stretchable tubular device 1 thus naturally retracts and releases its elastic energy, helping the heart efforts. The heart efforts may thus be assisted by more than 5%. During the systole, the decreased resistance of the tubular device 1 under the application of a voltage, limits the increase of the blood pressure, and thus limits the heart effort at this specific time. On the contrary, during the diastole, the wave of pressure is improved thanks to the natural elastic force F0, retrieved by the stretchable tubular device 1 when the applied voltage is decreased or shut off.

The level of voltage V may be determined according to the needs. A given stretchable tubular device 1 may be adapted for several different conditions. Depending on the necessary force modification F1 during the systole, a predetermined first level of voltage V1 may be determined. Alternatively, the modified force may be adaptable between a first modified force F1 and a second modified force F2 depending on the instant patient needs. A third modified force F3 may equally be determined. This also allows to tune the effect of the stretchable tubular device 1 once it is implanted in the body. Also, the duration of the voltage application may be object to fine adaptation.

The applied voltage may be, alternatively or in addition, instantaneously adapted to the actual need of the patent. Depending of the physical activity of the patient, or the cardiac rhythm, or several other physiological parameters and any combination thereof, a first V1 or a second V2 or a third V3 level of voltage may be instantaneously applied. For example, a maximal voltage level may be applied so as to decrease the force of the stretchable tubular device 1 to zero, during the systole. In this case, there is no increase of blood pressure and thus no additional effort from the heart. The voltage may be completely shut off at the diastole period so as to provide a maximal elastic force from the stretchable tubular device 1. An intermediate voltage may alternatively apply during the diastole so as to not deliver the maximal elastic force from the stretchable tubular device 1. Any combination of voltage may thus be determined. When used as such, the power supply 2 may be connected to a control unit (not shown) adapted to instantaneously determine the suitable voltage level to apply to the stretchable tubular device 1. Sensors may also be used to sense one or several physiological parameters related to the actual heart activity. Pressure sensors may alternatively or in addition be used for determining the pressure of the liquid B flowing through the stretchable tubular device 1.

Thus, the present invention allows for a method of helping the heart effort, in particular when the heart effort should be minimized during the systole period. The method using the stretchable tubular device further helps increasing the blood pressure at the diastole moment, by releasing the natural elastic energy of the stretchable tubular device 1.

An ensemble or a kit comprising several different stretchable devices 1 may also be used. As an example, a kit may comprise a first stretchable tubular device (1) having a natural force (F0) and at least a first modified force (F1) and at least a second stretchable tubular device (1′) having a natural force (F0′) and at least a first modified force (F1′). One or more of the natural force (F0′) and the modified force (F1′) of the second stretchable tubular device (1′) may differ from the natural force (F0) and/or the modified force (F1) of the first stretchable tubular device (1). Depending on the actual needs a particular stretchable tubular device of the kit may be selected according to its elastic properties.

Such a stretchable tubular device 1 may be used elsewhere than the aorta. A kit comprising several devices 1 may provide a first stretchable tubular device 1 adapted for the aorta, close to the heart. It may in addition offer at least one other stretchable tubular device having different properties, and dedicated to another site, or another artery.

The present disclosure also encompasses a method of regulating the pressure of a flowing liquid B. When flowing through the stretchable tubular device 1, the pressure of the flowing liquid B may be instantaneously adapted and regulated by the application of one or more different voltages to the stretchable tubular device 1. The method of regulating the pressure of a flowing liquid B can be applied to regulate the blood pressure. The stretchable tubular device 1 may thus be used as a counterpulsation device. To this end, it may be arranged on the aorta, and activated according to the following method, comprising the steps of:
Determining a first instant t1 on which a first level of voltage V1 is applied to the stretchable device 1, so as to induce a first modified force F1, lower than its natural force F0.
The first level of voltage V1 may be either the maximal applicable voltage, adapted to reduce the force of the stretchable tubular device 1 to zero. It can alternatively be an intermediate voltage adapted to partly reduce the force of the stretchable tubular device 1. The first instant t1 may be predetermined or determined based on one or more sensor activation. To this end a sensor may be used to determine the instantaneous blood pressure or the instantaneous cardiac contraction. The first instant t1 can for example correspond to the beginning of the systole period, or correspond to a moment before the systole period.

The method of regulating the pressure of a flowing liquid B may further comprise a second step of determining a second instant t2 on which a second level of voltage V2 is applied to the stretchable tubular device 1, so as to induce a second modified force F2. The second instant t2 may correspond to the beginning of the diastole period or to a moment shortly before the diastole starts or to a moment shortly after the diastole starts. The second instant t2 may be predetermined or determined according to instantaneous sensor data allowing to determine the instantaneous blood pressure or the instantaneous cardiac contractions. The second level of voltage V2 may be lower than the first level of voltage V1. It can also be completely null, in a way that the stretchable tubular device 1 retrieves its natural force F0 and provide a maximal elastic force.

The method of regulating the pressure of a flowing liquid B thus comprises at least two phases, a first phase starting at the first instant t1 wherein a first voltage V1 is applied, and ending at the beginning of the second phase, and a second phase starting at the second instant t2, wherein a second voltage V2 is applied. The second phase may end at the beginning of the first phase so as to repeat and alternate the first and the second phases. One or both of these two phases may comprise the application of one or more intermediate voltages.

According to a specific embodiment, the method of use of the stretchable tubular device 1 comprises a first phase starting at the first instant t1 wherein two intermediate voltages V1 and V1′ are alternatively applied several times so as to produce a fast pulsation during this first phase, and a second phase starting at the second instant t2 wherein a constant voltage V2 is applied. In particular, an alternance of two different voltages may be provided during the systole and the voltage may be switched off at the beginning of the diastole.

According to another embodiment, the method of use of the stretchable tubular device 1 comprises a first phase starting at the first instant t1 wherein a first constant voltage V1 is applied during a period shorter than the duration of the first phase, after which at least one intermediate constant voltage V1′ is applied before the end of the first phase. Said second constant voltage may last until the end of the first phase, after the second instant t2. Such a constant intermediate voltage V1′ allows to regulate the stretching of the device 1 when the blood pressure increases. In addition or alternatively, several different intermediate voltages may be successively applied during the first phase, until a final value, so as to progressively regulate the stretching of the stretchable tubular device 1 when the blood pressure decreases. The voltage may be switched off at the second instant t2 or at another moment during the second phase. Different timing and voltage combinations may be considered depending on the needs.

The layers Lx of the stretchable tubular polymer 1 may comprise any electroactive polymer or mixture of electroactive polymer. For a specific medical implant application, the layers Lx is preferably constituted of bio-compatible polymers such as silicone or other known bio-compatible polymers. Additional layers than those of stretchable polymer may be included in the stretchable device 1. For example, adhesion layers La may be used to maintain the stack of layers Lx and the corresponding electrodes 3a, 3b together. The arrangement of the layers comprised in the stretchable tubular device 1 is open to a variety and optimisation known to one skilled in the art. The layers Lx of the stretchable tubular device 1 may be for example as described in the patent application CH20190001382, without being limited to its disclosure.

The stretchable tubular device 1 may in addition comprise one or more than one shield layer Ls, having an electrical shield effect so as to avoid or limit potential leakage of current. This may be particularly important in case the stretchable tubular device 1 is used as an implant. The shield layer Ls is preferably arranged on top of the layers Lx, so as to isolate the stack of layers Lx from the outer space So of the stretchable tubular device 1. The shield layer Ls may be a conductive layer such as a carbon layer or any other conductive material suitable for an implant.

Although the stretchable tubular device 1 may be connected or may comprise one or more sensor adapted to determine some physiological parameters, such as the blood pressure, the presently disclosed device 1 can also be, alternatively or in addition, used as a sensor of such physiological parameters. A self-sensing function thus allows to better control the device and its instantaneous command.

The stretchable tubular device 1 may be produced according to the following sequence of steps.
S1: Depositing a conductive layer Lc on the top surface of the first layer L1. Such a deposited conductive layer may be a carbon layer, deposited by known methods such as vapor deposition or coating. The conductive layer Lc may alternatively be a conductive gel or any other suitable material. The first layer L1 may be free or combined with a first support layer Lz on its surface opposite to the top surface provided with the conductive layer Lc. The first support Lz may be made of PET or with other suitable polymer. The first layer may be for example of the type of Elastosil. The combination of the first layer L1 and the conductive layer Lc, and possibly the first support layer Lz forms a stack.
S2: Placing an adhesion film La1 on a second support layer Lz′. The second support layer Lz′ may be in the same material as the first support layer Lz or in a different material. It is preferably made in PET. Such adhesion layer La1 may be a custom-made carbone based layer using LSR4305.
S3: Placing the stack of layers obtained in the step S1 on the top of the adhesion layer La1 arranged in step S2, wherein the stack of layers is placed up-side down, meaning that the conductive layer Lc of the stack of layers is placed in contact with the adhesion layer La1. The layers are thus stacked so as to alternate the conductive layers Lc and the stretchable layers Lx.
The previous steps S1, S2 and S3 are iterated as many times as necessary, until the desired number of layers Lx is obtained. To this end, another adhesion layer La2 can be placed on the top of the stack of layers resulting from the previous sequence step S1, S2 and S3. In case a support layer Lz is used, it is removed before placing such adhesion layer La2 so that it is in direct contact with the surface of the stretchable layer L1. A new stack of layers is organized according to step S1 and then placed up-side down on the newly added conductive layer La2.

Once the number of layers Lx is adequate, a further step S4 of cutting the edges of the stacked layers is provided and a step S5 of a final deposition of a silicon layer so as to glue the stacked layers. Before the final deposition is made, the second support layer Lz′ may be removed. This step S5 results in a final stack of layer SL. The contact of the layers glued during step S5 will then be maintained during the following steps and in particular during the rolling step S6. The resulting final stack of layers SL of step S5 may have a parallelepiped shape having a longitudinal dimension comprised between around 5 to 30 cm, or 10 to 20 cm, and a transversal dimension comprised between around 2 and 10 cm, or between 3 and 5 cm.

In a step S6, the final stack of layers SL previously obtained is rolled around a tube T and the edges of the tube T are cut in a step S7 to allow electrical connection between the conductive layers and an external electrical device. The tube T may have a diameter comprised between 25 and 35 mm. It may for example be of around 30 mm diameter, corresponding to the diameter of the aorta at rest. The length of the tube T may be comprised between 30 and 80 mm, preferably between 50 and 70 mm. The tube T may be made or comprise a bio compatible polymer. It may be for example in PMMA. In a step S8, a layer of conductive silicone Lcs is deposited in order to establish the electrical contact with all the electrodes 3a, 3b and a wire is introduced. In further steps S9 and S10, one or more of inner and outer insulation layer Li is provided. The sequence of the steps S6 to S10 allows to provide the tubular shape of the device 1. After this sequence of steps or at any time in between, the adhesion of the layers, in the final stack of layers SL, may be checked by microscopic analysis.

Once the tubular shape is formed and isolated, a further step S11 of depositing a shielding layer Ls is operated by deep coating. The tubular shape may be immersed to this end in a reservoir of carbon ink. Then the necessary electrical connections are provided in a step S12. These electrical connexions include the connexion toward a power supply. This also includes the connexion of the shielding layer Ls to the ground.

It is highlighted that although the flowing liquid B described above preferably denotes the blood, it may denote any other liquid flowing through a tubular structure. The stretchable tubular device 1 is thus adapted to any system comprising a pump which pulses a liquid through a tubular structure. The stretchable tubular device 1 takes benefits of any increase of pressure of the flowing liquid, which is pulsed by a pump, and provides an additional contractile force due to the variation of its viscoelasticity. When the liquid is blood, the corresponding pump is the heart.

It is further highlighted that the voltage applied to the stretchable tubular device 1 has no direct incidence or no substantial direct incidence on its diameter. In other words, the applied voltage modifies the viscoelasticity of the stretchable tubular device without modifying or substantially modifying its diameter.

The modulation of the force applied by the stretchable tubular device exclusively depends on the voltage applied to it. In other words, the compression force provided by the stretchable tubular device 1 does not involve any additional mechanical elements such as springs or balloon.

It is thus understood that the stretchable tubular device 1 here described does not comprise additional mechanical parts such as spring or balloon. The variation of its diameter is exclusively due to the variation of pressure of the liquid flowing through it. Although it is preferably used without additional mechanical part, it may be combined with one or more of such mechanical parts allowing to actively modify its diameter. In this specific case, only its viscoelastic properties are modulated under application of a voltage, as described above.

1. Stretchable counterpulsation tubular device adapted for passively mimic the natural properties of an artery through which blood flows, comprising at least one layer of a stretchable polymer, a power supply and a set of electrodes connected to said power supply, wherein the power supply is adapted to supply at least a first level of voltage to said electrodes, wherein said tubular counterpulsation device has a native diameter and an inherent visco-elasticity exerting a natural force directed toward its inner space when its diameter is extended to a diameter by the expending force of the blood larger than its native diameter, and in that it has at least a first level of modified visco-elasticity when a first level of voltage is supplied to the layers of stretchable polymer through the electrodes, said first level of modified visco-elasticity exerting a first modified force directed toward the inner space of the tubular device when its diameter is extended to a diameter by the expending force of the blood larger than its native diameter, the first modified force being lower than the natural force.

2. Stretchable counterpulsation tubular device according to claim 1 wherein said at least one first voltage has no incidence on the diameter of the stretchable tubular device.

3. Stretchable counterpulsation tubular device according to claim 1, wherein said stretchable counterpulsation tubular device is exempt from mechanical parts such as springs and balloons.

4. (canceled)

5. Stretchable counterpulsation tubular device according to claim 1, wherein the thickness of the at least one layer is comprised between around 10 μm and around 500 μm, such as around 50 μm, 100 μm, 200 μm or 300 μm.

6. Stretchable counterpulsation tubular device according to claim 1, wherein the electrodes are made with carbon, and wherein the at least one layer of polymer is made with silicone and it is comprised between two electrodes.

7. Stretchable counterpulsation tubular device according to claim 1, wherein the number of layers is comprised between 2 and 50, forming a stack of layers, wherein each of the layers is comprised between two electrodes.

8. Stretchable counterpulsation tubular device according to claim 1, wherein the at least one layer has a thickness of around 100 μm, a capacitance of around 1.6 nF and a resistance of around 1 kΩ and wherein the first voltage is around 6 kV, or wherein the at least one layer has a thickness of around 200 μm, a capacitance of around 0.8 nF and a resistance of 10 kΩ and wherein the first voltage is around 12 kV.

9. Stretchable counterpulsation tubular device according to claim 1, being adapted to surround or replace a portion of a tubular structure such as an artery.

10. Stretchable counterpulsation tubular device according to claim 1, further comprising a sensor adapted to detect or determine an increase of the internal pressure of a flowing liquid passing through the tubular device.

11. Stretchable counterpulsation tubular device according to claim 1, further comprising a shielding layer.

12. Stretchable counterpulsation tubular device according to claim 1, further comprising a control unit, connected to the power supply, adapted to apply a given level of voltage at determined times and for determined periods of time and/or by intermittence.

13. Kit of several stretchable counterpulsation tubular devices, adapted for passively mimic the natural properties of an artery through which blood (B) flows, as described in claim 1, said kit comprising a first stretchable tubular device having a natural force and at least a first modified force and at least a second stretchable tubular device having a natural force and at least a first modified force, wherein at least one of the natural force and the modified force of the second stretchable tubular device differs from the natural force and the modified force of the first stretchable tubular device.

14. Process for producing the stretchable counterpulsation tubular device adapted for passively mimic the natural properties of an artery through which blood (B) flows, as defined in claim 1, comprising step sequence of producing several combinations of a stretchable layers and a conductive layer, and stacking said combinations so as to alternated the stretchable layers and conductive layers, resulting in a final stack of stretchable layers, and rolling said final stack of stretchable layers around a tube to produce a tubular shape, so that the diameter of said tubular shape of final stack of stretchable layers does not vary under the application of a voltage.

15. Process according to claim 13, further comprising a step of coating the obtained tubular shape with a conductive material to provide a shielding layer.

16. Stretchable counterpulsation tubular device, adapted for passively mimic the natural properties of an artery through which blood flows, according to claim 11, wherein said control unit, is adapted to:
apply at least one first voltage to the stretchable tubular device at a first instant, so as to induce a first modified force lower than its natural force;
apply at least one second voltage to the stretchable tubular device at a second instant, so as to induce at least a second modified force different than the first modified force;
wherein the first instant corresponds to the start of a first phase and the second instant corresponds to the start of a second phase and the end of the first phase, and wherein the first and the second phases are alternatively repeated.

17. Stretchable counterpulsation tubular device according to claim 15, wherein said stretchable tubular device is adapted to regulate the flowing of blood, and wherein said stretchable tubular device surrounds or replaces a portion of the aorta, said first phase corresponding to the systole and said second phase corresponding to the diastole, wherein during the first phase two intermediate voltages are alternatively applied several times or two or more constant voltages are successively applied.

18. Stretchable counterpulsation tubular device according to claim 15, wherein the voltage applied at the second instant or close to this second instant is null and remains null until the start of the first phase.
